(19) United States
(12) Patent Application Publication
(10) Pub. No.: US 2025/0387318 A1
(43) Pub. Date: Dec. 25, 2025

(54) VOLCANIC MUD OIL-CONTROLLING FACE-CLEANSING FREEZE-DRIED COSMETIC, AND PREPARATION METHOD AND APPLICATION THEREOF

(71) Applicant: Moya (Shanghai) Brand Management Co., Ltd., Shanghai (CN)

(72) Inventors: Kai Zhang, Lianyungang (CN); Gangbo Jin, Taizhou (CN)

(21) Appl. No.: 18/823,787

(22) Filed: Sep. 4, 2024

(30) Foreign Application Priority Data
Jun. 25, 2024 (CN) 2024108241461

(51) Int. Cl. A61K 8/96; A61K 8/02; A61K 8/34; A61K 8/44; A61K 8/73; A61Q 1/02; A61Q 5/02; A61Q 19/10

(52) U.S. Cl. CPC A61K 8/965; A61K 8/0212; A61K 8/345; A61K 8/442; A61K 8/732; A61Q 1/02; A61Q 5/02; A61Q 19/10

(57) ABSTRACT

The present invention discloses a volcanic mud oil-controlling face-cleansing freeze-dried cosmetic, and a preparation method and an application thereof, relating to the field of cosmetics, specifically including the following components: volcanic mud, cassava starch, corn starch, sodium lauroyl glutamate, and glycerol. The present invention perfectly arranges and combines the effective raw materials of traditional cosmetics and natural volcanic mud raw materials, which can allow targeted removal of excess oil, toxins, cosmetic residues and metal residues in the epidermis and hair follicles of oily skin, metabolize irreparable inflammatory cells through rejection reactions, stimulate the generation of new healthy cells, and realize skin cleansing, oil controlling, detoxification and repair, and at the same time, this invention utilizes vacuum freeze-drying technology to remove most of water added in products and retain the activity of active substances such as biological factors, which is beneficial to keep the freshness and stability of materials. Compared with traditional production methods, this vacuum freeze-drying technology avoids the problems of adding preservatives to control the oxidation, bacteria and mildew of raw materials, increases the shelf life of such cosmetics, and facilitates the transportation and carrying of the products.

### FIELD OF TECHNOLOGY

The present invention belongs to the field of cosmetic technology, and specifically to a volcanic mud oil-controlling face-cleansing freeze-dried cosmetic, and a preparation method and an application thereof.

### BACKGROUND

Sebum is one of the important substances in the skin. The production of sebum is continuous and not controlled by neural mechanisms. The amount of sebum produced is about 1 to 2 grams per 24 hours. However, the amount of sebum produced varies from person to person and is the origin of different skin types. In fact, this lipid secretion is a process controlled by various mechanisms, and its effects on the skin are as follows: a moisturizing effect, a protective effect, and a repairing effect;

the focus here is on ambient temperature; and ambient temperature directly affects changes in body temperature;

body temperature: a study has shown that temperature fluctuations can affect sebum secretion. In fact, every 1° C. increase/decrease in skin temperature leads to an increase/decrease of about 10% in sebum secretion. This may reflect changes in the secretion rate of sebum on the skin surface, which may be due to changes in sebum viscosity with temperature;

when excessive sebum secretion occurs, skin problems can occur. In fact, follicular sebaceous glands can block skin pores or hair roots, resulting in: greasy hair, clogged and dilated pores, and blemishes such as blackheads, pimples or acne, and folliculitis;

the above content cites: Cunliffe WJ, Burton JL, Shuster S. The effect of local temperature variations on the sebum excretion rate, British Journal Of Dermatology, 1970; 83(6):650-4;

a Sebumeter measuring instrument was used to monitor the facial sebum secretion of volunteers. In order to ensure the accuracy and consistency of the data, the sebum secretion

volume was measured in five different parts of the same volunteer's face (forehead, nose, chin, and cheeks) in different seasons of the year; and

the research results showed that there were significant differences in facial sebum secretion in different parts and seasons, and the specific findings were as follows:

regional differences:

the sebum secretion volume was higher in the forehead, nose, and chin (collectively referred to as T-zone) than in both cheeks (U-zone).

this phenomenon indicated that the T-zone was a more active area for sebum secretion, while the U-zone was relatively less active.

seasonal variations:

summer was a season with the highest sebum secretion volume, especially in the T-zone.

the lowest sebum secretion volume was observed in winter, with a more significant difference in the sebum secretion volume between the T-zone and U-zone.

in multiple seasons, the sebum secretion volume in the T-zone and U-zone were inconsistent.

### Conclusion

Research showed that facial sebum secretion not only varied in different zones, but also fluctuated with seasonal variations. Specifically, the secretion volume in the T zone significantly increased in summer, while the variation in the U-zone was relatively small. This pattern helped to develop more accurate skin care products and plans;

this research literature confirmed the influence of ambient temperature and seasonal variations on sebum secretion, providing a solid scientific basis for the present invention to develop a technology of adjusting oil control ingredients based on ambient temperature;

and

most of oil-controlling face-cleansing products on the market attempt to solve or alleviate problems such as pimples, acne, enlarged pores, and dull skin caused by excessive oil production with one product, ignoring the important impact of temperature changes on skin oil secretion. The present invention discloses a volcanic mud oil-controlling face-cleansing freeze-dried cosmetic, and a preparation method and an application thereof from two aspects, i.e., adjustment of product oil-controlling components according to ambient temperature (ATCOPCI)+the self-developed and innovative TCF-MVFM technology of a volcanic mud microporous vacuum freezing molding production process (VM-MVFM technology).

### SUMMARY

In view of the above situation, in order to overcome the defects of the prior art, the present invention provides a volcanic mud oil-controlling face-cleansing freeze-dried cosmetic, and a preparation method and an application thereof, effectively solving the problems proposed in the background.

To achieve the above purpose, the present invention provides the following technical solutions: a volcanic mud oil-controlling face-cleansing freeze-dried cosmetic, specifically including the following components by mass percentage: 24% of volcanic mud; 22.4% of cassava starch; 22.4% of corn starch; 20% of sodium lauroyl glutamate; 6% of glycerol; 2.2% of a trimethylpentanediol/adipic acid/glycerol cross-linked polymer; 1% of panthenol; 0.45% of diisostearyl malate; 0.4% of ascorbic acid; 0.4% of cajeput oil; 0.25% of chondrus crispus; 0.15% of potassium chloride; 0.1% of xanthan gum; 0.03% of dihydroxypropylarginine hydrochloride; 0.001% of 1,2 pentanediol; 0.1% of sodium chloride; and 0.115% of water.

Preferably, one or more mixed raw material ingredients of the volcanic mud, the corn starch, the cassava starch, the sodium glutamate, and the like are included.

Preferably, one of or a mixture of more than one of the sodium lauroyl glutamate, the glycerol, the trimethylpentanediol/adipic acid/glycerol cross-linked polymer, the panthenol, the diisostearyl malate, the ascorbic acid, the cajeput oil, the chondrus crispus, the potassium chloride, the xanthan gum, the dihydroxypropylarginine hydrochloride, and the sodium chloride is included.

A preparation method of a volcanic mud oil-controlling face-cleansing freeze-dried cosmetic, specifically including the following steps:
1. device preparation: preparing one clean stirring container and stirrer, one clean homogenizer, and one clean heating device;
2. raw material preparation:
   1) weighing a desired water-soluble raw material;
   2) weighing desired diisostearyl malate and cajeput oil;
   3) each of them through a sieve to remove impurities and lumps to ensure that powder is fine and uniform; and
   4) preparing a special silicone mold;
3. water preparation: using deionized water or purified water to ensure quality and purity of water, where a ratio by weight of total water to total raw materials desired is 45:55 according to a formula, that is, 450 grams of water is needed for 550 grams of the raw materials, and gradually adding the raw materials according to characteristics and dissolution sequence of each raw material, where an ingredient with a larger usage amount is first added, and then an ingredient with a smaller usage dosage is sequentially added; and

specific operations
S1. adding deionized water or purified water weighed according to the ratio into a stirring container, starting to heat with a temperature controlled at 70° C.-80° C., gradually adding a trimethylpentanediol/adipic acid/glycerol cross-linked polymer into a heated aqueous phase, and slowly stirring to ensure continuous stirring until the polymer is completely dissolved to form a uniform solution;
S2. gradually adding sodium lauroyl glutamate, glycerol, panthenol, chondrus crispus, potassium chloride, xanthan gum, dihydroxypropylarginine hydrochloride, 1,2-pentanediol, and sodium chloride when a mixture is cooled to a temperature of 30° C.-40° C., and thoroughly stirring after adding a raw material each time to ensure that the raw material is completely dissolved;
S3. adding an oily ingredient: for diisostearyl malate, slowly adding into the mixture, and stirring at a low speed with a rotating speed set in a range of 300-500 RPM to ensure that the diisostearyl malate is uniformly dispersed; and

for cajeput oil, adding into the aqueous phase using a same method, and stirring at the same low speed to ensure that the cajeput oil is uniformly dispersed;
S4. cooling the mixture to 20° C.-25° C.; and continuously stirring during a cooling process to prevent ingredients from precipitation or agglomeration;
S5. cooling the mixture to 4° C.-10° C., and under dark conditions, adding ascorbic acid into water, and quickly stirring to ensure that the ascorbic acid is completely dissolved;
S6. one homogenization treatment

transferring the mixture to the homogenizer for treatment; and setting a rotating speed of the homogenizer to 1000-3000 RPM and homogenizing time to 10-15 minutes, and ensuring that a temperature is maintained in a range of 20° C.-30° C. during a homogenization process;
S7. gradually adding the volcanic mud powder, the corn starch and the cassava starch into the aqueous phase mixture, and stirring at a slow speed of about 200-500 RPM to ensure that powder raw materials are uniformly dispersed in the aqueous phase mixture;
S8. secondary homogenization treatment: setting the rotating speed of the homogenizer to 2000 RPM for secondary homogenization treatment and homogenizing time to 15 minutes;
S9. adjustment and packaging

after completing the stirring and homogenization treatment, cooling the mixture to room temperature; and

detecting uniformity and stability of the final mixture, and after confirming its qualification, transferring it to a container for further use;
S10. rapid freeze molding:

injecting the above aqueous mixture into a prepared

silicone mold with a tray, then entering a freezing machine together with the tray, and setting a temperature to −40° C. and time to 2 h-4 h;
S11. granule separation screening

observing freeze molding condition of the mixture, and after reaching an existing standard, turning on the freezing machine, take outing the silicone tray, taking out the silicone mold at the same time, putting the silicone mold into a mold rolling machine for granule separation, detecting integrity of granules, and putting the complete frozen granules into the tray;
S12. drying and dehydration

primary drying stage:

under low temperature and low pressure conditions, directly sublimating ice in the cosmetic into water vapor to separate the ice from a solid substance;

temperature setting: slowly increasing from −30° C. to −40° C. to −10° C. to 0° C., and vacuum pressure: 0.3 mbar;

time setting: 15 h; and

resolution drying stage:

further removing residual moisture from the cosmetic to ensure that a product is completely dry;

temperature: slowly increasing from 0° C. to 40° C.;

pressure: maintained at 0.01 Pa; and

time: 5 hours; and
S13. packaging, recording and storage

subpackaging: removing the product from the freezing machine, and subpackaging it into final retail packaging.

Preferably, a packaging specification of the cosmetic after preparation is 0.4 g/granule and 40 granules/bottle.

A use method of a volcanic mud oil-controlling face-cleansing freeze-dried cosmetic, including first moistening a face with clear water, taking one granule of a product in a palm, dissolving the product with a small amount of water, rubbing the product to foam, evenly applying the product to the face, massaging the face for 10 seconds, and then thoroughly cleaning the face.

An application of a volcanic mud oil-controlling face-cleansing freeze-dried cosmetic, including an application of the volcanic mud according to any one of claims as a color makeup, a facial mask and shampoo, which has a cleansing function and a beauty function of controlling oil and repairing damaged skin cells, making skin or scalp balanced in water and oil and full of elasticity and show a healthy and youthful posture.

The technical effects and advantages of the present invention:
1. the present invention perfectly arranges and combines the effective raw materials of traditional cosmetics and natural volcanic mud raw materials, which can allow targeted removal of excess oil, toxins, cosmetic residues and metal residues in the epidermis and hair follicles of oily skin, metabolize irreparable inflammatory cells through rejection reactions, stimulate the generation of new healthy cells, and realize skin cleansing, oil controlling, detoxification and repair, and at the same time, this invention utilizes vacuum freeze-drying technology to remove most of water added in products and retain the activity of active substances such as biological factors, which is beneficial to keep the freshness and stability of materials. Compared with traditional production methods, this vacuum freeze-drying technology avoids the problems of adding preservatives to control the oxidation, bacteria and mildew of raw materials, increases the shelf life of such cosmetics, and facilitates the transportation and carrying of the products.

### DESCRIPTION OF THE EMBODIMENTS

The present invention provides a volcanic mud oil-controlling face-cleansing freeze-dried cosmetic, specifically including the following components by mass percentage: 24% of volcanic mud; 22.4% of cassava starch; 22.4% of corn starch; 20% of sodium lauroyl glutamate; 6% of glycerol; 2.2% of a trimethylpentanediol/adipic acid/glycerol cross-linked polymer; 1% of panthenol; 0.45% of diisostearyl malate; 0.4% of ascorbic acid; 0.4% of cajeput oil; 0.25% of chondrus crispus; 0.15% of potassium chloride; 0.1% of xanthan gum; 0.03% of dihydroxypropylarginine hydrochloride; 0.001% of 1,2 pentanediol; 0.1% of sodium chloride; and 0.115% of water.

It should be noted that one or more mixed raw material ingredients of the volcanic mud, the corn starch, the cassava starch, the sodium glutamate, and the like are included.

It should also be noted that one of or a mixture of more than one of the sodium lauroyl glutamate, the glycerol, the trimethylpentanediol/adipic acid/glycerol cross-linked polymer, the panthenol, the diisostearyl malate, the ascorbic acid, the cajeput oil, the chondrus crispus, the potassium chloride, the xanthan gum, the dihydroxypropylarginine hydrochloride, and the sodium chloride is included.

A preparation method of a volcanic mud oil-controlling face-cleansing freeze-dried cosmetic, specifically including the following steps:
1. device preparation: preparing one clean stirring container and stirrer, one clean homogenizer, and one clean heating device;
2. raw material preparation:
   1) weighing a desired water-soluble raw material.
   2) weighing desired diisostearyl malate and cajeput oil;
   3) each of them through a sieve to remove impurities and lumps to ensure that powder is fine and uniform. Note: If the raw material has a high water content, appropriate drying treatment is required to prevent agglomeration during a mixing process;
   4) preparing a special silicone mold. Note: non-toxic and harmless: a silicone material must be non-toxic and harmless and meet the safety standards of the cosmetics industry to ensure no side effects on the human body; high purity: the purity of the material should be high to avoid affecting or contaminating ingredients of the cosmetic;

stability: silicone needs to remain stable under different temperatures and humidity conditions, without degradation, discoloration, deterioration, and other phenomena; good flexibility: in a freeze-drying process, silicone must be able to maintain good flexibility and shape, avoiding cracking or deformation during use; easy to clean: silicone should be easy to clean and disinfect for reuse, reducing costs; anti-static performance: silicone needs to have a good anti-static performance to prevent the adsorption of dust or other small particles during production and use; chemical corrosion resistance: silicone should have strong resistance to chemical substances such as cosmetic ingredients and detergents, avoiding chemical reactions with them;

heat and cold resistance: in a production process of the freeze-dried cosmetic, silicone needs to withstand temperatures ranging from −60° C. to 200° C. without degradation, deformation or deterioration; and

compliance with relevant regulations: the silicone material must comply with relevant national or international regulations and standards in the cosmetics industry;
3. water preparation: using deionized water or purified water to ensure quality and purity of water. A ratio by weight of total water to total raw materials desired is 45:55 according to a formula, that is, 450 grams of water is needed for 550 grams of the raw materials, and gradually adding the raw materials according to characteristics and dissolution sequence of each raw material, where an ingredient with a larger usage amount is first added, and then an ingredient with a smaller usage dosage is sequentially added; and specific operations
S1. adding deionized water or purified water weighed according to the ratio into a stirring container, starting to heat with a temperature controlled at 70° C.-80° C., gradually adding a trimethylpentanediol/adipic acid/glycerol cross-linked polymer into a heated aqueous phase, and slowly stirring to ensure continuous stirring until the polymer is completely dissolved to form a uniform solution;
S2. gradually adding sodium lauroyl glutamate, glycerol, panthenol, chondrus crispus, potassium chloride, xanthan gum, dihydroxypropylarginine hydrochloride, 1,2-pentanediol, and sodium chloride when a mixture is cooled to a temperature of 30° C.-40° C., and thoroughly stirring after adding a raw material each time to ensure that the raw material is completely dissolved;
S3. adding an oily ingredient: for diisostearyl malate, slowly adding into the mixture,

and stirring at a low speed with a rotating speed set in a range of 300-500 RPM to ensure that the diisostearyl malate is uniformly dispersed; and

for cajeput oil, adding into the aqueous phase using a same method, and stirring at the same low speed to ensure that the cajeput oil is uniformly dispersed;
S4. cooling the mixture to 20° C.-25° C.; and continuously stirring during a cooling process to prevent ingredients from precipitation or agglomeration;
S5. cooling the mixture to 4° C.-10° C., and under dark conditions, adding ascorbic acid into water, and quickly stirring to ensure that the ascorbic acid is completely dissolved;
S6. one homogenization treatment

transferring the mixture to the homogenizer for treatment; and setting a rotating speed of the homogenizer to 1000-3000 RPM and homogenizing time to 10-15 minutes, and ensuring that a temperature is maintained in a range of 20° C.-30° C. during a homogenization process;
S7. gradually adding the volcanic mud powder, the corn starch and the cassava starch into the aqueous phase mixture (maintained at a temperature between 20° C. and 25° C.), and stirring at a slow speed of about 200-500 RPM to ensure that powder raw materials are uniformly dispersed in the aqueous phase mixture;
S8. secondary homogenization treatment: setting the rotating speed of the homogenizer to 2000 RPM for secondary homogenization treatment and homogenizing time to 15 minutes;
S9. adjustment and packaging

after completing the stirring and homogenization treatment, cooling the mixture to room temperature; and

detecting uniformity and stability of the final mixture, and after confirming its qualification, transferring it to a container for further use;
S10. rapid freeze molding:

injecting the above aqueous mixture into a prepared

silicone mold with a tray, then entering a freezing machine together with the tray, and setting a temperature to −40° C. and time to 2 h-4 h;
S11. granule separation screening

observing freeze molding condition of the mixture, and after reaching an existing standard, turning on the freezing machine, take outing the silicone tray, taking out the silicone mold at the same time, putting the silicone mold into a mold rolling machine for granule separation, detecting integrity of granules, and putting the complete frozen granules into the tray;
S12. drying and dehydration

primary drying stage (sublimation drying):

under low temperature and low pressure conditions, directly sublimating ice in the cosmetic into water vapor to separate the ice from a solid substance;

temperature setting: slowly increasing from −30° C. to −40° C. to −10° C. to 0° C., and vacuum pressure: 0.3 mbar;

time setting: 15 h; and

resolution drying stage (secondary drying):

further removing residual moisture from the cosmetic to ensure that a product is completely dry;

temperature: slowly increasing from 0° C. to 40° C.;

pressure: maintained at about 0.01 Pa; and

time: generally 5 hours; and
S13. packaging, recording and storage

subpackaging: removing the product from the freezing machine, and subpackaging it into final retail packaging.

It should also be noted that a packaging specification of the cosmetic after preparation is 0.4 g/granule and 40 granules/bottle.

A use method of a volcanic mud oil-controlling face-cleansing freeze-dried cosmetic, including first moistening a face with clear water, taking one granule of a product in a palm, dissolving the product with a small amount of water, rubbing the product to foam, evenly applying the product to the face, massaging the face for 10 seconds, and then thoroughly cleaning the face.

An application of a volcanic mud oil-controlling face-cleansing freeze-dried cosmetic, including an application of the volcanic mud according to any one of claims as a color makeup, a facial mask and shampoo, which has a cleansing function and a beauty function of controlling oil and repairing damaged skin cells, making skin or scalp balanced in water and oil and full of elasticity and show a healthy and youthful posture.

It should be noted that relational terms such as first and second herein are only used to distinguish one entity or operation from another entity or operation, and do not necessarily require or imply that any such actual relationship or order exists between these entities or operations. Moreover, the terms “including”, “comprising” or any other variation thereof are intended to encompass non-exclusive inclusion, such that a process, method, article, or apparatus including a series of elements not only includes those elements, but also includes other elements not explicitly listed, or also includes elements inherent to such a process, method, article, or apparatus.

Although the embodiments of the present invention have been shown and described, it can be understood by those of ordinary skill in the art that multiple variations, modifications, substitutions, and variations can be made to these embodiments without departing from the principles and spirit of the present invention, the scope of which is defined by the appended claims and their equivalents.

1. A volcanic mud oil-controlling face-cleansing freeze-dried cosmetic, specifically comprising the following components: volcanic mud, cassava starch, corn starch, sodium lauroyl glutamate, and glycerol.

2. The volcanic mud oil-controlling face-cleansing freeze-dried cosmetic according to claim 1, specifically comprising the following components by mass percentage: 24% of the volcanic mud; 22.4% of the cassava starch; 22.4% of the corn starch; 20% of the sodium lauroyl glutamate; 6% of the glycerol; 2.2% of a trimethylpentanediol/adipic acid/glycerol cross-linked polymer; 1% of panthenol; 0.45% of diisostearyl malate; 0.4% of ascorbic acid; 0.4% of cajeput oil; 0.25% of chondrus crispus; 0.15% of potassium chloride; 0.1% of xanthan gum; 0.03% of dihydroxypropylarginine hydrochloride; 0.001% of 1,2 pentanediol; 0.1% of sodium chloride; and 0.115% of water.

3. The volcanic mud oil-controlling face-cleansing freeze-dried cosmetic according to claim 2, comprising one or more mixed raw material ingredients of the volcanic mud, the corn starch, the cassava starch, the sodium glutamate, and the like.

4. The volcanic mud oil-controlling face-cleansing freeze-dried cosmetic according to claim 3, comprising one of or a mixture of more than one of the sodium lauroyl glutamate, the glycerol, the trimethylpentanediol/adipic acid/glycerol cross-linked polymer, the panthenol, the diisostearyl malate, the ascorbic acid, the cajeput oil, the chondrus crispus, the potassium chloride, the xanthan gum, the dihydroxypropylarginine hydrochloride, and the sodium chloride.

5. A preparation method of a volcanic mud oil-controlling face-cleansing freeze-dried cosmetic, specifically comprising the following steps:
1. device preparation: preparing one clean stirring container and stirrer, one clean homogenizer, and one clean heating device;
2. raw material preparation:
1) weighing a desired water-soluble raw material;
2) weighing desired diisostearyl malate and cajeput oil;
3) weighing desired volcanic mud powder, corn starch and cassava starch, and passing each of them through a sieve to remove impurities and lumps to ensure that powder is fine and uniform; and
4) preparing a special silicone mold;
3. water preparation: using deionized water or purified water to ensure quality and purity of water, wherein a ratio by weight of total water to total raw materials desired is 45:55 according to a formula, that is, 450 grams of water is needed for 550 grams of the raw materials, and gradually adding the raw materials according to characteristics and dissolution sequence of each raw material, wherein an ingredient with a larger usage amount is first added, and then an ingredient with a smaller usage dosage is sequentially added; and
specific operations
S1. adding deionized water or purified water weighed according to the ratio into a stirring container, starting to heat with a temperature controlled at 70° C.-80° C., gradually adding a trimethylpentanediol/adipic acid/glycerol cross-linked polymer into a heated aqueous phase, and slowly stirring to ensure continuous stirring until the polymer is completely dissolved to form a uniform solution;
S2. gradually adding sodium lauroyl glutamate, glycerol, panthenol, chondrus crispus, potassium chloride, xanthan gum, dihydroxypropylarginine hydrochloride, 1,2-pentanediol, and sodium chloride when a mixture is cooled to a temperature of 30° C.-40° C., and thoroughly stirring after adding a raw material each time to ensure that the raw material is completely dissolved;
S3. adding an oily ingredient: for diisostearyl malate, slowly adding into the mixture, and stirring at a low speed with a rotating speed set in a range of 300-500 RPM to ensure that the diisostearyl malate is uniformly dispersed; and
for cajeput oil, adding into the aqueous phase using a same method, and stirring at the same low speed to ensure that the cajeput oil is uniformly dispersed;
S4. cooling the mixture to 20° C.-25° C.; and continuously stirring during a cooling process to prevent ingredients from precipitation or agglomeration;
S5. cooling the mixture to 4° C.-10° C., and under dark conditions, adding ascorbic acid into water, and quickly stirring to ensure that the ascorbic acid is completely dissolved;
S6. one homogenization treatment
transferring the mixture to the homogenizer for treatment; and setting a rotating speed of the homogenizer to 1000-3000 RPM and homogenizing time to 10-15 minutes, and ensuring that a temperature is maintained in a range of 20° C.-30° C. during a homogenization process;
S7. gradually adding the volcanic mud powder, the corn starch and the cassava starch into the aqueous phase mixture, and stirring at a slow speed of about 200-500 RPM to ensure that powder raw materials are uniformly dispersed in the aqueous phase mixture;
S8. secondary homogenization treatment: setting the rotating speed of the homogenizer to 2000 RPM for secondary homogenization treatment and homogenizing time to 15 minutes;
S9. adjustment and packaging
after completing the stirring and homogenization treatment, cooling the mixture to room temperature; and
detecting uniformity and stability of the final mixture, and after confirming its qualification, transferring it to a container for further use;
S10. rapid freeze molding:
injecting the above aqueous mixture into a prepared
silicone mold with a tray, then entering a freezing machine together with the tray, and setting a temperature to −40° C. and time to 2 h-4 h;
S11. granule separation screening
observing freeze molding condition of the mixture, and after reaching an existing standard, turning on the freezing machine, take outing the silicone tray, taking out the silicone mold at the same time, putting the silicone mold into a mold rolling machine for granule separation, detecting integrity of granules, and putting the complete frozen granules into the tray;
S12. drying and dehydration
primary drying stage:
under low temperature and low pressure conditions, directly sublimating ice in the cosmetic into water vapor to separate the ice from a solid substance;
temperature setting: slowly increasing from −30° C. to −40° C. to −10° C. to 0° C., and vacuum pressure: 0.3 mbar;
time setting: 15 h; and
resolution drying stage:
further removing residual moisture from the cosmetic to ensure that a product is completely dry;
temperature: slowly increasing from 0° C. to 40° C.;
pressure: maintained at 0.01 Pa; and
time: 5 hours; and
S13. packaging, recording and storage
subpackaging: removing the product from the freezing machine, and subpackaging it into final retail packaging.

6. The preparation method of a volcanic mud oil-controlling face-cleansing freeze-dried cosmetic according to claim 5, wherein a packaging specification of the cosmetic after preparation is 0.4 g/granule and 40 granules/bottle.

7. A use method of a volcanic mud oil-controlling face-cleansing freeze-dried cosmetic, comprising first moistening a face with clear water, taking one granule of a product in a palm, dissolving the product with a small amount of water, rubbing the product to foam, evenly applying the product to the face, massaging the face for 10 seconds, and then thoroughly cleaning the face.

8. An application of a volcanic mud oil-controlling face-cleansing freeze-dried cosmetic, comprising an application of the volcanic mud according to claim 1 as a color makeup, a facial mask and shampoo, which has a cleansing function and a beauty function of controlling oil and repairing damaged skin cells, making skin or scalp balanced in water and oil and full of elasticity and show a healthy and youthful posture.

9. An application of a volcanic mud oil-controlling face-cleansing freeze-dried cosmetic, comprising an application of the volcanic mud according to claim 2 as a color makeup, a facial mask and shampoo, which has a cleansing function and a beauty function of controlling oil and repairing damaged skin cells, making skin or scalp balanced in water and oil and full of elasticity and show a healthy and youthful posture.

10. An application of a volcanic mud oil-controlling face-cleansing freeze-dried cosmetic, comprising an application of the volcanic mud according to claim 3 as a color makeup, a facial mask and shampoo, which has a cleansing function and a beauty function of controlling oil and repairing damaged skin cells, making skin or scalp balanced in water and oil and full of elasticity and show a healthy and youthful posture.

11. An application of a volcanic mud oil-controlling face-cleansing freeze-dried cosmetic, comprising an application of the volcanic mud according to claim 4 as a color makeup, a facial mask and shampoo, which has a cleansing function and a beauty function of controlling oil and repairing damaged skin cells, making skin or scalp balanced in water and oil and full of elasticity and show a healthy and youthful posture.
